(19) United States
(12) Patent Application Publication
(10) Pub. No.: US 2009/0137024 A1
(43) Pub. Date: May 28, 2009

(54) Method of Separating Target DNA from Mixed DNA

(75) Inventor: Michele R. Stone, Rockville, MD (US)

Correspondence Address: ROTHWELL, FIGG, ERNST & MANBECK, P.C., 1425 K STREET, N.W., SUITE 800, WASHINGTON, DC, 20005, US

(73) Assignee: CANON U.S. LIFE SCIENCES, INC., Rockville, MD (US)

(21) Appl. No.: 11/946,549

(22) Filed: Nov. 28, 2007

(51) Int. Cl. C12N 1/08

(52) U.S. Cl. 435/270

(57) ABSTRACT

The present invention relates to methods of separating target DNA from mixed DNA in a sample. In some embodiments, the target DNA may be viral DNA, bacterial DNA, fungal DNA or combinations thereof. In some embodiments the mixed DNA includes target DNA and non-target DNA.

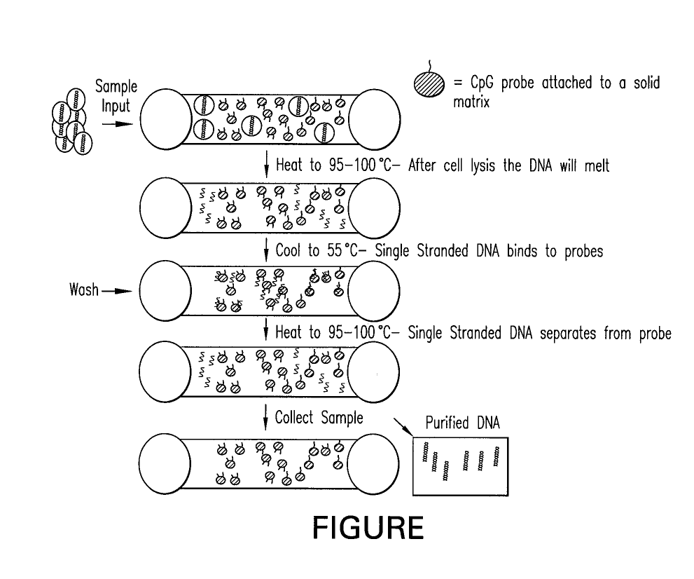

### BACKGROUND

1. Field of the Invention

The present invention relates to methods of separating target DNA from mixed DNA in a sample. In some embodiments, the target DNA may be viral DNA, bacterial DNA, fungal DNA or combinations thereof. In some embodiments the mixed DNA includes target DNA and non-target DNA.

2. Description of Related Art

The detection of nucleic acids is central to medicine. The ability to detect infectious organisms (e.g., viruses, bacteria, fungi) is ubiquitous technology for disease diagnosis and prognosis. Determination of the integrity of a nucleic acid of interest can be relevant to the pathology of an infection. One of the most powerful and basic technologies to detect small quantities of nucleic acids is to replicate some or all of a nucleic acid sequence many times, and then analyze the amplification products. PCR is perhaps the most well-known of a number of different amplification techniques. The nucleic acids are generally isolated from a sample prior to detection, although in situ detection can also be performed.

The basic steps of nucleic acid, such as DNA, isolation are disruption of the cellular structure to create a lysate, separation of the soluble nucleic acid from cell debris and other insoluble material, and purification of the DNA of interest from soluble proteins and other nucleic acids. Historically, organic extraction (e.g., phenol:chloroform) followed by ethanol precipitation was done to isolate DNA. Disruption of most cells is done by chaotropic salts, detergents or alkaline denaturation, and the resulting lysate is cleared by centrifugation, filtration or magnetic clearing. The DNA can then be purified from the soluble portion of the lysate. When silica matrices are used, the DNA is eluted in an aqueous buffer such as Tris-EDTA (TE) or nuclease-free water.

DNA isolation systems for genomic, plasmid and PCR product purification are historically based on purification by silica. Regardless of the method used to create a cleared lysate, the DNA of interest can be isolated by virtue of its ability to bind silica in the presence of high concentrations of chaotropic salts (Chen and Thomas, Anal Biochem 101:339-341, 1980; Marko et al., Anal Biochem 121:382-387, 1982; Boom et al., J Clin Microbiol 28:495-503, 1990). These salts are then removed with an alcohol-based wash and the DNA eluted in a low ionic strength solution such as TE buffer or water. The binding of DNA to silica seems to be driven by dehydration and hydrogen bond formation, which competes against weak electrostatic repulsion (Melzak et al., J Colloid and Interface Science 181:635-644, 1996). Hence, a high concentration of salt will help drive DNA adsorption onto silica, and a low concentration will release the DNA.

Recently, new methods for DNA purification have been developed which take advantage of the negatively charged backbone of DNA to a positively charged solid substrate (under specific pH conditions), and eluting the DNA using a change in solvent pH (ChargeSwitch® technology, Invitrogen, Corp., Carlsbad, Calif.; see, for example, U.S. Pat. No. 6,914,137 and International Published Application No. 2006/004611). Whatman has an alternate technology (FTA® paper) that utilizes a cellulose based solid substrate impregnated with a lysis material that lyses cells, inactivates proteins, but captures DNA in the cellulose fibers, where it is retained for use in downstream applications (see, for example, U.S. Pat. No. 6,322,983).

In addition, a significant problem with the above technologies is that they require the use of specific buffers for DNA binding and washing. Most of these buffers are not compatible with downstream applications, such as PCT. These technologies also have a wide range of efficiencies in the overall quantity of DNA that is purified. Regardless of the applications there is no way to use any of the above described technologies to separate (or enrich for) viral, bacterial or fungal DNA from (over) mammalian DNA. A method that would require no specific buffers for lysis or binding to the solid matrix is not commercially available.

Early detection of infectious agents in a mammalian tissue sample, such as whole blood, requires that a few infectious agent DNA molecules be detected in a background of many mammalian tissue DNA molecules. Separation of the infectious agent DNA molecules from the mammalian tissue DNA molecules would improve detection efficiencies by lowering the background of mammalian DNA in the sample. None of the above described methods address the problem of purifying bacterial, viral, or fungal DNA separately from mammalian DNA in a mixed DNA sample. Thus, a need exists for methods that provide for the enrichment and purification of viral, bacterial or fungal DNA in the presence of mammalian DNA.

### SUMMARY OF THE INVENTION

The present invention relates to methods for separating target DNA from non-target DNA in a sample. In some embodiments, the target DNA may be viral DNA, bacterial (or prokaryotic) DNA, fungal DNA or combinations thereof. In some embodiments, the non-target DNA is mammalian DNA.

Thus, in a first aspect, the present invention provides a method of separating target DNA from mixed DNA in a sample comprising: (a) contacting a sample comprising target DNA and non-target DNA with an agent that binds target DNA but does not bind non-target DNA, (b) separating the target DNA from the non-target DNA and (c) recovering the target DNA from the binding agent. In some embodiments, the target DNA may be viral DNA, bacterial DNA, fungal DNA and combinations thereof. In some embodiments, the non-target DNA is mammalian DNA. In some embodiments, the sample is contacted with the agent for a length of time sufficient to bind the target DNA. In other embodiments, the agent is attached to a solid substrate. In some embodiments, the agent is a probe containing one or more CpG motifs that are selective for target DNA. In other embodiments, the agent is a combination of probes which may contain the same or different CpG motifs or may contain a polymeric CpG motif.

In further embodiments, the sample comprises cells and the method further comprises first lysing the cells before contacting the sample with the agent. In some embodiments, the lysis is performed by chemical lysis. In other embodiments, the lysis is performed by mechanical energy, such as electric, pressure, acoustic, homogenization and freeze thawing. In additional embodiments, the lysis is performed by heat. In further embodiments, the method further comprises removing cellular debris from the lysed sample prior to contacting with the agent. In some embodiments, the target DNA and non-target DNA is rendered single-stranded. In other embodiments, the contacting is performed at a temperature in which the single-stranded target DNA binds to the binding agent, e.g., probe. In some embodiments, the separation is performed by removing the non-target DNA from the solid substrate containing the bound target DNA. In other embodiments, the non-target DNA is removed by washing. In some embodiments, the solid substrate is a magnetic bead, a matrix, a particle, a polymeric bead, a chromotagraphic resin, filter paper, a membrane or a hydrogel.

In a second aspect, the present invention provides a method of separating target DNA from mixed DNA in a cellular sample comprising: (a) lysing the cells of a cellular sample comprising target DNA and non-target DNA, (b) removing cellular debris from the lysed sample, (c) contacting the lysed sample with an agent that binds target DNA but does not bind non-target DNA, and (d) separating the target DNA from the non-target DNA. In some embodiments, the target DNA may be viral DNA, bacterial DNA, fungal DNA and combinations thereof. In other embodiments, the non-target DNA is mammalian DNA. In some embodiments, the lysis is performed by chemical lysis. In other embodiments, the lysis is performed by mechanical energy. In further embodiments, the lysis is performed by heat. In other embodiments, the sample is contacted with the agent for a length of time sufficient to bind the target DNA. In other embodiments, the agent is attached to a solid substrate. In some embodiments, the agent is a probe containing one or more CpG motifs that are selective for target DNA.

In further embodiments, the method further comprises removing cellular debris from the lysed sample prior to contacting with the agent. In some embodiments, the target DNA and non-target DNA is rendered single-stranded. In other embodiments, the contacting is performed at a temperature in which the single-stranded target DNA binds to the binding agent, e.g., probe. In some embodiments, the separation is performed by removing the non-target DNA from the solid substrate containing the bound target DNA. In other embodiments, the non-target DNA is removed by washing. In some embodiments, the solid substrate is a magnetic bead, a matrix, a particle, a polymeric bead, a chromotagraphic resin, filter paper, a membrane or a hydrogel.

The above and other embodiments of the present invention are described below with reference to the accompanying drawings.

### BRIEF DESCRIPTION OF THE DRAWING

The accompanying drawing, which is incorporated herein and forms part of the specification, illustrates the present invention.

The FIGURE shows an illustration of separating mammalian DNA from bacterial DNA in accordance with an embodiment of the present invention

### DETAILED DESCRIPTION OF THE INVENTION

The present invention has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, Oligonucleotide Synthesis: A Practical Approach, 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

As described above, there are no methods which address the problem of purifying bacterial, viral, and/or fungal DNA separately from mammalian DNA in a mixed DNA sample. The present invention provides for the enrichment and purification of bacterial, viral and/or fungal DNA in the presence of mammalian DNA. Thus, the present invention relates to methods for separating target DNA from non-target DNA in a mixed DNA sample.

The present invention provides for the separation of non-target DNA, e.g., mammalian DNA, from target DNA, e.g., bacterial, viral and/or fungal DNA, by utilizing distinguishing characteristics of target DNA and non-target DNA with respect to their CpG motifs. Vertebrate genomic DNA is pervasively CpG suppressed. The traditional explanation for this centers on methylation of CpG dinucleotides at position 5 of the cytosine base, which through deamination of 5-methylcytosine (possibly enzymatically mediated) and failure to repair the mismatch, mutates to TpG/CpA. At least 60% of CpG in some sequences in vertebrate DNA is methylated. The CpG dinucleotide relative abundance is normal in almost all invertebrate and fungal, and most common bacteria. The CpG motif is 100 times more prevalent in the prokaryotic genome than in the eukaryotic genome. Thus, this distinction is used by the present invention to enrich the bacterial DNA over mammalian DNA.

Thus, in a first aspect, the present invention provides a method of separating target DNA from mixed DNA in a sample comprising: (a) contacting a sample comprising target DNA and non-target DNA with an agent that binds target DNA but does not bind non-target DNA, (b) separating the target DNA from the non-target DNA and (c) recovering the target DNA from the binding agent. In some embodiments, the target DNA is viral DNA, bacterial DNA, fungal DNA or combinations thereof. In some embodiments, the non-target DNA is mammalian DNA. In some embodiments, the sample is contacted with the agent for a length of time sufficient to bind the target DNA. In other embodiments, the agent is attached to a solid substrate. In some embodiments, the agent is a probe containing one or more CpG motifs that are selective for target DNA.

The binding agent is capable of binding to target DNA, for example, viral, bacterial and/or fungal DNA, but does not preferentially bind to non-target DNA, for example, mammalian DNA. In a preferred embodiment, the target DNA is bacterial DNA. In one embodiment, the binding agent is one or more probes that bind to CpG motifs commonly found in the target DNA but not commonly found in the non-target DNA. Such probes are sometimes referred to herein as CpG probes. The target and non-target DNA is treated to render it single-stranded using techniques well known in the art. In one embodiment, the sample is heated to 95°-100° C. for a sufficient length of time to melt all of the DNA in the sample, i.e., to render all of the DNA as single-stranded DNA.

The CpG motif found predominantly in microorganisms is comprised of a sequence containing a core CpG flanked by two 5′ purines (A, G) and two 3′ pyrimidines (T, C). This motif is rarely found in eukaryotic DNA due to the methylation of the cytosine in the CpG core and the spontaneous mutation of the C to a T, due to deamination. Spontaneous deamination of dimethylcytosine results in thymine and ammonia. In DNA, this reaction cannot be corrected because the repair mechanisms do not recognize thymine as erroneous (as opposed to uracil), and unless it affects the function of the gene, the mutation will persist. This flaw in the repair mechanism contributes to the rarity of CpG sites in the eukaryotic genome.

Taking advantage of the sequence bias in microorganisms it would be possible to enrich a DNA sample for prokaryotic DNA vs eukaryotic DNA. This mechanism allows for the specific segregation of microbial DNA versus mammalian DNA. The sequences set forth in Table 1 are possible combinations of the CpG motif.

TABLE 1

GGCGTT AACGTT GACGTT AGCGTT
GGCGCC AACGCC GACGCC AGCGCC
GGCGTC AACGTC GACGTC AGCGTC
GGCGCT AGCGCC AGCGTC AGCGCT

These CpG motifs can be used alone or in combination together or with other sequence to bind specifically to the microbial DNA vs mammalian DNA along the microbial genome. The probe can contain one or more of the CpG motifs or can contain multiple copies of a CpG motif. The length of the probe can vary and suitable probe lengths are well known to the skilled artisan. See, for example, U.S. Pat. No. 4,358,535, Crosa et al. (J Bact 115:904-911, 1973), Keller and Manak (DNA Probes, Stockton Press, New York, 1989) and Ausubel et al. (Current Protocols in Molecular Biology (John Wiley & Sons, New York, 1992, including periodic updates). In some embodiments, the probes are attached to a solid substrate. In some embodiments, each probe is a single CpG motif. One or more of the same or different probes of this embodiment can be individually attached to the solid substrate. In other embodiments, each probe is a multiple polymeric CpG motif or combination of CpG motifs. One or more of the same of different probes of this embodiment can be individually attached to the solid substrate. In additional embodiments, each probe contains a sequence that includes one or more CpG motifs. One or more of the same or different probes of this embodiment can be individually attached to the solid substrate. In further embodiments, any combinations of these probes can be individually attached to the solid substrate.

The “solid substrate” or “solid phase” or “solid matrix” is not critical and can be selected by one skilled in the art. A “solid phase”, as used herein, refers to any material which is insoluble, or can be made insoluble by a subsequent reaction. Any known solid support may be used. Examples of commonly used solid phase materials include, but are not limited to, matrices, particles, micro beads and macro beads free in solution, made of any known material, e.g., nitrocellulose, nylon, glass, polyacrylates, mixed polymers, polystyrene, silane polypropylene, silica gel, metal, such as paramagnetic particles. See, for example, U.S. Pat. Nos. 4,358,535, 4,797,355, 5,237,016, 7,214,780 and 7,294,489. In some embodiments, the solid substrate may include a magnetic bead, a matrix, a particle, a polymeric bead, a chromotagraphic resin, filter paper, a membrane or a hydrogel. Among the advantages of solid phase systems is that the reaction product or products can be washed with relative ease to remove the non-target DNA.

Methods for the immobilization of probes are well known to those skilled in the art. Suitable methods for immobilizing probes on solid phases include ionic, hydrophobic, covalent interactions, chelation and the like. For example, a probe may be immobilized by adsorption to a solid phase or by covalent attachment to a solid phase. Alternatively, a probe may be immobilized indirectly by one or more linkers. The manner of coupling a probe to a solid phase material is known. See, for example, U.S. Pat. Nos. 4,358,535, 4,797,355, 4,806,546, 5,237,016, 5,252,724, 7,214,780 and 7,294,489. Alternatively, a probe may be tagged with a small molecule such as biotin and either avidin or an antibody to biotin may be immobilized on a solid phase.

In other embodiments, the sample comprises cells and the method further comprises first lysing the cells before contacting the sample with the agent. In some embodiments, the lysis is performed by chemical lysis. In other embodiments, the lysis is performed by mechanical energy. In further embodiments, the method further comprises removing cellular debris from the lysed sample prior to contacting with the agent.

Commercial cell lysis products can be used to lyse cells in the cellular sample. Such commercial cell lysis products include, but are not limited to, Poppers Cell Lysis Reagents (Pierce, Rockville, Ill., USA), Wizard® Genomic DNA Purification Kit (Promega Corp., Madison, Wis., USA), lysis solutions from Qiagen, Inc. (Valencia, Calif., USA), and Cell Lysis Solution (Spectrum Chemical and Laboratory Products, Gardena, Calif., USA).

Alternatively, mechanical energy, preferably acoustic energy, can be used to lyse cells in a cellular sample. Any device that generates a sound wave can be used as a source of acoustic energy for lysing the cells. Such devices include, but are not limited to, ultrasonic transducers, piezoelectric transducers, magnorestrictive transducers and electrostatic transducers. Suitable devices are well known in the art including such commercially available devices as Sonicator 4000 (Misonix, Inc., Farmingdale, N.Y., USA), Microson® Sonicator Microprobe or Micro Cup Horn (Kimble/Kontes, Vineland, N.J., USA) and Covaris™ Adaptive Focused Acoustics (Nexus Biosystems, Poway, Calif., USA). Other suitable devices are described in U.S. Pat. Nos. 6,881,541 and 6,878,540 and in U.S. Patent Application Publication No. 2007/0170812. One advantage of lysing cells using mechanical energy is that not only are the cells lysed, but the DNA is also sheared to generate fragments of DNA. It is easier for the binding agents, to interact with the DNA of smaller fragments.

In other embodiments, the cells are lysed as part of the step that includes contacting the target DNA with the binding agent. In one embodiment, the cells are lysed by heating to a temperature sufficient to render the DNA in the sample as single-stranded DNA. The sample is heated for a length of time sufficient to lyse the cells, typically, 2-5 minutes.

In some embodiments, the non-target DNA is removed by washing the bound target DNA. For example, the immobilized target DNA can be washed with water or simple buffers to remove contaminates, inhibitors (of downstream processing applications) and non-target DNA. The elution mixture can be anything from water to PCR buffer, whatever is compatible with downstream analysis methods.

In some embodiments, the target DNA is recovered and collected. In one embodiment, an elution volume is added the sample after the non-target DNA has been removed. The sample is then heated to a temperature, e.g., up to 95°-100° C., to denature the target DNA-probe complex. The purified target DNA is recovered and collected for downstream

One embodiment of the invention allows for the removal of mammalian DNA from the cell lysate and thus allows for the enrichment of bacterial, viral and/or fungal DNA over the background of host DNA. This allows for increased signal to noise ratio in molecular diagnostic assays (example PCR reactions), which is important in cases where it is necessary to detect rare targets, such as bacteria, viruses or fungi.

The present invention can be practiced using readily available materials as described above to separate target DNA and non-target DNA in a mixed DNA sample.

In one embodiment, the solid matrix CpG probe is mixed with the lysed sample. The temperature is increased to 95°-100° C. and incubated for various lengths of time depending on the sample being processed in order to denature the target DNA and the non-target DNA present in the sample. Standard methods may be used to lyse the mammalian cells and microorganisms present in the sample. Then the sample is cooled to 55° C. to allow the probes to bind to various regions along the target DNA, thereby binding the target DNA to a solid matrix. The immobilized target DNA can then be washed with water or simple buffers to remove contaminates, inhibitors (of downstream processing applications) and non-target DNA. The elution mixture can be anything from water to PCR buffer, whatever is compatible with downstream analysis methods. Once the elution volume is added the sample is then heated up to 95°-100° C. to denature the target DNA-probe complex. The purified target DNA is recovered and collected for downstream applications.

The FIGURE is an illustration of another embodiment of the present invention. As shown in the FIGURE, one or more CpG probes are bound to a solid phase. A sample and the CpG containing solid matrix are added to a reaction zone. The sample is heated to 95°-100° C. and incubated for 2 minutes to lyse the cells and microorganisms present in the sample and to render the target DNA and non-target DNA that has been liberated from the cells single-stranded. The temperature is lowered to 55° C. for the single-stranded target DNA to bind to the CpG DNA sequences that are covalently attached to a solid matrix. Once the target DNA is bound, the mixture is washed to remove all contaminants including non-target DNA. The sample is then heated again to 95°-100° C. to release the target DNA from the CpG matrix. The purified target DNA is recovered and collected for downstream processing.

In a second aspect, the present invention provides a method of separating target DNA from mixed DNA in a cellular sample comprising: (a) lysing the cells of a cellular sample comprising target DNA and non-target DNA, (b) removing cellular debris from the lysed sample, (c) contacting the lysed sample with an agent that binds target DNA but does not bind non-target DNA, and (d) separating the target DNA from the non-target DNA. In some embodiments, the target DNA may be viral DNA, bacterial DNA, fungal DNA and combinations thereof. In a preferred embodiment, the target DNA is bacterial DNA. In other embodiments, the non-target DNA is mammalian DNA. In some embodiments, the lysis is performed by chemical lysis as described herein. In other embodiments, the lysis is performed by mechanical energy, preferably acoustic energy, as described herein. In further embodiments, the lysis is performed by heating the sample as described herein. In other embodiments, the sample is contacted with the agent for a length of time sufficient to bind the target DNA. In other embodiments, the agent is attached to a solid substrate as described herein. In some embodiments, the agent is a probe containing one or more CpG motifs that are selective for target DNA as described herein.

In further embodiments, the method further comprises removing cellular debris from the lysed sample prior to contacting with the agent as described herein. In other embodiments, the contacting is performed at a temperature in which the single-stranded target DNA binds to the binding agent, e.g., probe. In some embodiments, the separation is performed by removing the non-target DNA from the solid substrate containing the bound target DNA. In other embodiments, the non-target DNA is removed by washing as described herein. In some embodiments, the solid substrate is a solid substrate as described herein and may include a magnetic bead, a matrix, a particle, a polymeric bead, a chromotagraphic resin, filter paper, a membrane or a hydrogel.

The current state of the art in molecular diagnostics for infectious disease does not include separation of bacterial, viral and/or fungal DNA from background mammalian DNA in tissue extracts. Instead the mixed sample is utilized for the specific amplification and detection of the target bacterial, viral and/or fungal DNA. In many cases the background mammalian DNA interferes with amplification and detection. The present invention can be used to remove background mammalian DNA prior to the amplification and detection steps of diagnostic procedures for the bacterial, viral and/or fungal DNA.

An advantage of the methods of the present invention is that microbial DNA can be selectively enriched over eukaryotic DNA (100:1). The wash buffer and elution buffer can be any reagent that is compatible with down stream applications. The simplicity and speed of this method is also a significant advantage.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of separating target DNA from non-target DNA comprising:
contacting a sample comprising target DNA and non-target DNA with an agent that binds target DNA but does not bind non-target DNA, wherein the target DNA is selected from the group consisting of viral DNA, bacterial DNA, fungal DNA and combinations thereof;
separating the target DNA from the non-target DNA; and
recovering the target DNA from the binding agent.

2. The method of claim 1, wherein the agent is coupled to a solid substrate.

3. The method of claim 1, wherein the separation is performed by washing the non-target DNA from the bound target DNA.

4. The method of claim 2, wherein the separation is performed by washing the non-target DNA from the bound target DNA.

5. The method of claim 1, wherein the agent that binds target DNA is a probe containing a CpG motif that binds target DNA.

6. The method of claim 2, wherein the solid substrate is a magnetic bead, a matrix, a particle, a polymeric bead, a chromotagraphic resin, filter paper, a membrane or a hydrogel.

7. The method of claim 6, wherein the solid substrate is a matrix.

8. The method of claim 1, wherein the sample comprises cells and the method further comprises first lysing the cells before contacting the sample with the agent.

9. The method of claim 8, wherein the lysis is performed by chemical lysis.

10. The method of claim 8, wherein the lysis is performed by mechanical energy.

11. The method of claim 8, wherein the lysis is performed by heat.

12. The method of claim 8, which further comprises removing cellular debris from the lysed sample prior to contacting with the agent.

13. The method of claim 1, wherein the sample is contacted with the agent for a length of time sufficient to bind the target DNA

14. The method of claim 1, wherein the non-target DNA is mammalian DNA.

15. A method of separating target DNA from non-target DNA comprising:
heating a sample comprising target DNA and non-target DNA to a temperature sufficient to lyse cells in the sample and to render the target DNA and non-target DNA single-stranded, wherein the target DNA is selected from the group consisting of viral DNA, bacterial DNA, fungal DNA and combinations thereof;
contacting single-stranded target DNA and single-stranded non-target DNA sample with an agent that binds single-stranded target DNA but does not bind single-stranded non-target DNA;
separating the target DNA from the non-target DNA; and
recovering the target DNA from the binding agent.

16. The method of claim 15, wherein the agent is coupled to a solid substrate.

17. The method of claim 15, wherein the separation is performed by washing the non-target DNA from the bound target DNA.

18. The method of claim 16, wherein the separation is performed by washing the non-target DNA from the bound target DNA.

19. The method of claim 15 wherein the agent that binds single-stranded target DNA is a probe containing a CpG motif that binds single-stranded target DNA.

20. The method of claim 16, wherein the solid substrate is a magnetic bead, a matrix, a particle, a polymeric bead, a chromotagraphic resin, filter paper, a membrane or a hydrogel.

21. The method of claim 15, wherein the sample is contacted with the agent for a length of time sufficient to bind the target DNA

22. The method of claim 15, wherein the non-target DNA is mammalian DNA.

23. The method of claim 15, wherein the contacting step includes lowering the temperature to a temperature sufficient for the single-stranded target DNA to bind to the binding agent.

24. The method of claim 15, wherein the target DNA is recovered from the binding agent by heating the bound target DNA to a temperature sufficient to separate the target DNA from the binding agent.
